(19) United States
(12) Patent Application Publication
(10) Pub. No.: US 2026/0308350 A1
(43) Pub. Date: Oct. 8, 2026

(54) CYCLODEPOLYMERIZATION METHOD FOR AROMATIC POLYESTERS

(71) Applicant: SOOCHOW UNIVERSITY, Suzhou (CN)

(72) Inventors: Yingfeng TU, Suzhou (CN); Bilin Xiang, Suzhou (CN); Jing Li, Suzhou (CN); Xiaohong Li, Suzhou (CN); Zhipeng Zhao, Suzhou (CN); Shujie Huang, Suzhou (CN)

(21) Appl. No.: 19/456,621

(22) Filed: Jan. 22, 2026

Related U.S. Application Data
(63) Continuation of application No. PCT/CN2023/132874, filed on Nov. 21, 2023.

(30) Foreign Application Priority Data
Aug. 1, 2023 (CN) 202310954558.2

(51) Int. Cl. C08J 11/22; C07D 323/00; C08G 63/78

(52) U.S. Cl. CPC C08J 11/22; C07D 323/00; C08G 63/78

(57) ABSTRACT

The invention provides a cyclodepolymerization method for aromatic polyesters. The method includes mixing aromatic polyesters with a high-boiling-point green solvent and a catalyst, and heating and dissolving the mixture to subject the aromatic polyesters to a cyclodepolymerization reaction to obtain the corresponding cyclic oligoesters, wherein the high-boiling-point solvent is a green solvent that does not contain a halogen or an aromatic group, such as 1,3-dimethyl-2-imidazolinone, the heating temperature is 180-250° C., the catalyst is an organic base catalyst, and the yield of the separated and purified cyclic oligoesters can reach 91%. The method can be used for the chemical recycling of polyesters, by using a green and environmentally friendly solvent as the reaction solvent. The recycled aromatic polyester is depolymerized under the catalysis of the organic base catalyst to obtain the aromatic cyclic oligoesters, thereby achieving the green chemical recycling of aromatic polyesters.

### FIELD OF THE INVENTION

The present disclosure relates to the technical field of chemical depolymerization of polymer materials, and in particular to a cyclodepolymerization method for aromatic polyesters.

### DESCRIPTION OF THE RELATED ART

Globally, the increasing environmental protection awareness and the increasing shortage of petroleum resources have stimulated people's interest in a sustainable recycling economy in polymerization, and the recycling technology of polymer materials has become a research focus at home and abroad. Aromatic polyesters have excellent thermal and mechanical properties, among which ethylene terephthalate (PET) has excellent overall properties, and is widely used in the fields of packaging, electronics, fibers, and the like. The global yield of PET exceeded 70 million tons in 2019. Since aromatic polyesters such as PET have stable properties and are not easy to degrade, the pollution problem caused by the production and application of a large number of PET products is increasing. Therefore, the development of a simple and efficient green recycling method is of great significance for the recycling of waste aromatic polyesters. Chemical recycling refers to a recycling technology in which waste polyester is converted into oligomers or small molecule raw materials through chemical reactions, which are then polymerized to produce polyester again or converted into related petrochemicals, which belongs to the upcycling technology.

Taking PET as an example, chemical recycling can be divided into hydrolysis, alcoholysis, aminolysis, and the like. The hydrolysis refers to depolymerizing PET into terephthalic acid (or salt) and ethylene glycol in an acidic or alkaline aqueous solution under high-temperature and high-pressure conditions. The hydrolysis has the disadvantages that a large amount of water and acid or base compounds are needed, and the generated large amount of waste liquid must be treated; the acid or base compounds are easy to corrode equipment under the high-temperature and high-pressure conditions; Additionally, the presence of oligomer byproducts in the product poses a challenge for purification. The alcoholysis refers to subjecting PET to a transesterification reaction with an alcohol solvent (e.g., methanol or ethylene glycol) under appropriate temperature and pressure to obtain corresponding ester compounds (e.g., dimethyl terephthalate and diethylene terephthalate) and ethylene glycol. The alcoholysis has the disadvantages that a large amount of an alcohol solvent is required as a reactant and a solvent; the presence of oligomer byproducts in the product poses a challenge for purification. The aminolysis refers to the aminolysis reaction of PET in a primary amine solution such as ethylenediamine or ethanolamine to produce terephthalic acid diamide, such as bis(2-hydroxyethyl) terephthalic acid diamide. The aminolysis has the disadvantages of relatively long reaction time and relatively more side reactions.

In addition to chemical recycling routes such as hydrolysis, alcoholysis, and aminolysis, aromatic polyesters such as PET can also be subjected to cyclodepolymerization based on the principle of ring-chain equilibrium. Ring-chain equilibrium is a process of mutual transformation between linear and cyclic molecules controlled by thermodynamic equilibrium in the polymer synthesis process. By shifting the equilibrium from linear polyesters towards the formation of cyclic esters, cyclic oligoesters are obtained. In recent years, the preparation of cyclic oligoesters from linear polyesters by ring-chain equilibrium has gained a lot of attention in the industry. Unlike hydrolysis, alcoholysis, aminolysis and other methods, the cyclic oligoesters prepared by cyclodepolymerization are difficult to synthesize by ordinary reactions, and therefore have high technical barriers and added values.

Brunelle et al., in U.S. Pat. No. 5,668,186 (published on Sep. 16, 1999), disclosed a method for preparing aromatic cyclic oligoesters. By depolymerizing an aromatic polyester in the presence of an organic solvent such as orthodichlorobenzene using a titanium catalyst based on a total molar amount of polyester monomers of about 1-5% to obtain the corresponding aromatic cyclic oligoesters. However, PET demonstrates inadequate solubility in orthodichlorobenzene, and orthodichlorobenzene is a chlorine-containing aromatic solvent, which is carcinogenic and environmentally unfriendly. Shi Jiaxin et al., in Chinese Patent No. CN 102060983 A (published on May 18, 2011), disclosed a method for preparing aromatic cyclic oligoesters. By depolymerizing polyethylene terephthalate in the presence of high-boiling-point organic solvents such as orthodichlorobenzene, trichlorobenzene, and methylnaphthalene in the presence of an organic tin catalyst to obtain the corresponding cyclic oligoesters. However, the solvents such as orthodichlorobenzene and trichlorobenzene are chlorine-containing aromatic solvents, and methylnaphthalene is an aromatic solvent, which are carcinogenic and environmentally unfriendly. In addition, the cyclic stannoxane catalyst needs to be self-made, which makes industrial production difficult. Tu Yingfeng et al., in Chinese Patent No. CN 113735817 A (published on Dec. 3, 2021), disclosed a method for preparing an aliphatic macrocyclic oligoesters. The aliphatic polyester is depolymerized in the presence of toluene/xylene to obtain the corresponding aliphatic macrocyclic oligoesters, but the method is only suitable for aliphatic polyesters with good solubility in toluene/xylene, and toluene/xylene is an aromatic solvent and is carcinogenic. Therefore, in light of environmental considerations, it is necessary to adopt halogen-free and aromatic group-free environmentally friendly solvents for chemical recycling of aromatic polyesters.

In another aspect, the catalysts utilised in the aforementioned operations are all metal catalysts, which inevitably introduce metal ions into the system, thereby exerting an effect on the application of the cyclic oligoesters and giving rise to environmental concerns. In recent years, there has been a growing interest in organic catalysts, which facilitate chemical reactions through the utilisation of organic small molecules. Compared with metal catalysts, organic catalysts have the advantages of being less toxic, more environmentally friendly, and less sensitive to oxygen and moisture. The organic base catalyst can catalyze not only the ring-opening polymerization of a cyclic compound, but also the depolymerization of a polymer. However, there is currently no report on the use of organic base catalysts in the cyclodepolymerization reaction of aromatic polyesters.

Based on the aforementioned analysis, there is a need to develop a green and efficient cyclodepolymerization method for an aromatic polyester with an organic catalyst carried out in an environmentally friendly solvent. Since waste aromatic polyesters can be prepared into the corresponding cyclic aromatic oligoesters by this method, it is of great significance for the green chemical recycling of waste aromatic polyesters.

### SUMMARY OF THE INVENTION

The present disclosure provides a green cyclodepolymerization method for an aromatic polyester, the implementation of which is intended to resolve the technical problems described above. cyclodepolymerization In the method, aromatic polyesters such as PET are used as raw materials, a green and environment-friendly high-boiling-point solvent is used as a solvent, and cyclodepolymerization is performed under the catalysis of an organic base catalyst to obtain aromatic cyclic oligoesters with a high yield (the reaction route is shown in FIG. 1). When waste aromatic polyesters are used as raw materials, corresponding aromatic cyclic oligoesters can be prepared therefrom, so that green chemical recycling of waste aromatic polyesters is realized.

The present disclosure is implemented by the following technical solutions:

It is an object of the present disclosure to provide a cyclodepolymerization method for an aromatic polyester, including the following steps:
mixing an aromatic polyester with a high-boiling-point solvent and a catalyst to obtain a mixture. Subsequent heating and dissolving the mixture to subject the aromatic polyester to a depolymerization and cyclization reaction so as to obtain aromatic cyclic oligoesters, wherein the high-boiling-point solvent is selected from the group consisting of N-methylpyrrolidone (NMP), 1,3-dimethyl-2-imidazolinone (DMI), and 1,3-dimethyl-3,4,5,6-tetrahydro-2-pyrimidone (DMPU) and any combination thereof.

In the prior art, the solvent used for recycling an aromatic polyester by the cyclodepolymerization method is generally a high-boiling-point chlorine-containing or aromatic group-containing organic solvent such as 1,2-dichlorobenzene, 1-methylnaphthalene, diphenyl ether. Aromatic polyesters, such as PET, demonstrate limited solubility in the aforementioned solvents, 1,2-dichlorobenzene and 1-methylnaphthalene are carcinogenic, and diphenyl ether is toxic to aquatic organisms such as fish, which is not environmentally friendly.

In the present disclosure, green solvents with high boiling points and low toxicity, such as N-methylpyrrolidone, 1,3-dimethyl-2-imidazolinone, and 1,3-dimethyl-3,4,5,6-tetrahydro-2-pyrimidone, are used as reaction solvents. Such solvents do not contain a halogen or aromatic group, are strongly polar aprotic solvents, and have properties such as low melting points, low toxicity, high boiling points, strong solubility, and excellent stability. Such solvents can dissolve aromatic polyesters such as PET at high temperature, and also provide good solubility for an organic base catalyst, which can improve the reaction rate and yield of the cyclodepolymerization reaction. Such solvents have high boiling points and excellent stability at high temperature, and can be used for reactions at high temperature. The high temperature is advantageous for the cyclodepolymerization reaction under thermodynamic control, and the reaction solvent can be recycled for the cyclodepolymerization reaction.

In one embodiment of the present disclosure, the aromatic polyester is a recycled or synthetic aromatic polyester, so that green chemical recycling of waste aromatic polyesters can be achieved; the recycled aromatic polyester includes a recycled bottle chip and/or a recycled fiber.

In one embodiment of the present disclosure, the aromatic polyester is formed by polymerization of an aromatic diacid and an alkylene glycol, and has a structural formula as follows:

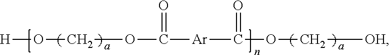

wherein Ar is an aromatic ring; a=2-4, and a is an integer; n≥4, and n is an integer.

In one embodiment of the present disclosure, the aromatic ring is a benzene ring and/or a furan ring; the alkylene glycol is selected from the group consisting of ethylene glycol, 1,3-propanediol, 1,4-butanediol and any combination thereof.

In one embodiment of the present disclosure, when the aromatic polyester described above is used as a reaction raw material, the aromatic cyclic oligoesters obtained by depolymerization is a mixture of aromatic cyclic oligoestess with different degrees of polymerization, and the aromatic cyclic oligoesters have a structural formula shown as follows:

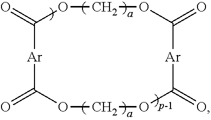

wherein Ar is an aromatic ring, a=2-4, p=2-13, and each of a and p is an integer.

In the prior art, the catalyst used for recycling an aromatic polyester by the cyclodepolymerization method is a metal catalyst, including tin, titanium, zinc, and the like. Metal catalysts have relatively high toxicity and are difficult to remove completely in the purification process.

In one embodiment of the present disclosure, the catalyst is an organic base catalyst; the organic base catalyst has relatively high catalytic efficiency and is environmentally friendly; the organic base catalyst is an alkylamine catalyst, a pyridine derivative catalyst, an amidine catalyst, a guanidine catalyst, an azacyclic carbene catalyst, or a phosphonitrile catalyst.

In one embodiment of the present disclosure, the alkylamine catalyst includes, but is not limited to 1,4-diazabicyclo[2.2.2]octane (DABCO) and 1-azabicyclo[2.2.2]octane (ABCO); the pyridine derivative catalyst includes, but is not limited to 4-dimethylaminopyridine (DMAP) and 4-pyrrolidinylpyridine (4-PPy); the amidine catalyst includes, but is not limited to 1,8-diazabicyclo[5,4,0]undecene-7 (DBU), 1,5-diazabicyclo[4.3.0]non-5-ene (DBN), and N-methylimidazole (NMI); the guanidine catalyst includes, but is not limited to 1,5,7-triazabicyclo[4.4.0]dec-5-ene (TBD), 7-methyl-1,5,7-triazabicyclo[4.4.0]dec-5-ene (MTBD), and tetramethylguanidine (TMG); the azacyclic carbene catalyst includes, but is not limited to 1,3-Bis(2,6-diisopropylphenyl) imidazolium chloride; the phosphonitrile catalyst includes, but is not limited to 2-tert-butylimino-2-diethylamino-1,3-dimethylperhydro-1,3,2-diazaphosphorine (BEMP).

In one embodiment of the present disclosure, the amount of the catalyst added has a certain influence on the yield of the target product. For the cyclodepolymerization reaction of an aromatic polyester, in a certain range, the more the catalyst is added, the faster the reaction rate is, and the higher the yield is at the same reaction time. The catalyst is added in an amount of 0.1%-10% by mass of the aromatic polyester, preferably 1%-5%.

In one embodiment of the present disclosure, the concentration of the aromatic polyester in the mixture of the present disclosure has a certain influence on the yield of the target product. In a certain range, the yield increases as the concentration of aromatic polyester decreases, that is, the cyclodepolymerization reaction of the aromatic polyester in the present disclosure is more suitable to be carried out under a dilute solution condition. The aromatic polyester has a concentration of 1 g/L to 100 g/L, preferably 5 g/L to 50 g/L.

In one embodiment of the present disclosure, the reaction is performed under an inert atmosphere.

In one embodiment of the present disclosure, the reaction time has a certain influence on the yield of the target product. In a certain range, the yield is increasingly high as the reaction time is prolonged. The reaction time in the present disclosure can be controlled at 3 h-24 h. Preferably, the reaction time is 8 h-16 h.

In one embodiment of the present disclosure, the reaction temperature has a certain influence on the yield of the target product. In a certain range, the yield is increasingly high as the reaction temperature increases. In the present disclosure, the reaction temperature can be controlled at 180° C.-250° C. Preferably, the reaction temperature is 220° C.-230° C.

In one embodiment of the present disclosure, the aromatic cyclic oligoesters obtained by cyclodepolymerization can be further polymerized as a monomer to produce an aromatic polyester.

Compared with the prior art, the above technical solutions of the present disclosure have the following advantages.
1. For the cyclodepolymerization method for an aromatic polyester of the present disclosure, the solvents used are green low-toxic high-boiling-point strongly polar organic solvents, N-methylpyrrolidone (NMP), 1,3-dimethyl-2-imidazolinone (DMI), and 1,3-dimethyl-3,4,5,6-tetrahydro-2-pyrimidone (DMPU). Such solvents provide good solubility for aromatic polyesters and catalysts at high temperature, have low toxicity, do not contain halogens or aromatic groups, can reduce environmental problems in industrial production, have excellent stability and high boiling points, can be recycled and reused, and reduce production costs.
2. For the cyclodepolymerization method for an aromatic polyester of the present disclosure, the catalyst used is an organic base catalyst, which has high catalytic activity for the cyclodepolymerization reaction of the aromatic polyester, and has low toxicity, easy to remove, more environmentally friendly, low sensitivity to oxygen and moisture, and diverse sources.
3. For the cyclodepolymerization method for an aromatic polyester of the present disclosure, the yield of cyclic oligoesters is significantly improved compared with that using a metal catalyst (see Examples 1-11 and Comparative Examples 1-3). Under optimal reaction conditions, the yield can be as high as 91% after purified by chromatographic separation and precipitation (see Example 15).
4. The cyclodepolymerization method for an aromatic polyester of the present disclosure can be used for chemically recycling waste aromatic polyesters and depolymerizing the waste aromatic polyesters into cyclic oligoesters, wherein the obtained cyclic oligoesters can be used as monomers for further polymerization reaction to give polyesters with high molecular weight and high added value. The method realizes the recycling of waste aromatic polyesters, thereby achieving the green recycling and high-value utilization of aromatic polyesters such as PET.

### BRIEF DESCRIPTION OF THE DRAWINGS

In order to facilitate a clear understanding of the contents of the present disclosure, the present disclosure will be further illustrated in detail below according to specific embodiments of the present disclosure in conjunction with the accompanying drawings. In the drawings,

FIG. 1 shows a reaction route of a cyclodepolymerization reaction of an aromatic polyester in the present disclosure;

FIG. 2 shows ¹H NMR spectra of cyclic oligomeric polyethylene terephthalate according to Example 1 of the present disclosure (solvent: deuterated trichloromethane);

FIG. 3 shows a MALDI-TOF MS spectrum of cyclic oligomeric polyethylene terephthalate according to Example 1 of the present disclosure, wherein signal peaks are sodium adduct molecular ion peaks of cyclic monomers with different degrees of oligomerization;

FIG. 4 shows a ¹H NMR spectra of cyclic oligomeric polypropylene terephthalate according to Example 23 of the present disclosure (solvent: deuterated trichloromethane);

FIG. 5 shows a MALDI-TOF MS spectrum of cyclic oligomeric polypropylene terephthalate according to Example 23 of the present disclosure, wherein signal peaks are sodium adduct molecular ion peaks of cyclic monomers with different degrees of oligomerization;

FIG. 6 shows ¹H NMR spectra of cyclic oligomeric butylene terephthalate according to Example 24 of the present disclosure (solvent: deuterated trichloromethane);

FIG. 7 shows a MALDI-TOF MS spectrum of cyclic oligomeric butylene terephthalate according to Example 24 of the present disclosure, wherein signal peaks are sodium adduct molecular ion peaks of cyclic monomers with different degrees of oligomerization;

FIG. 8 shows a ¹H NMR spectra of cyclic oligomeric polyethylene furandicarboxylate according to Example 25 of the present disclosure (solvent: deuterated dichloromethane);

FIG. 9 shows a MALDI-TOF MS spectrum of cyclic oligomeric polyethylene furandicarboxylate according to Example 25 of the present disclosure, wherein signal peaks are sodium adduct molecular ion peaks of cyclic monomers with different degrees of oligomerization.

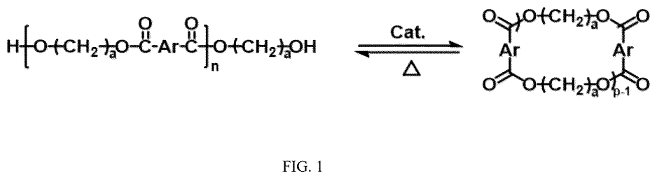

### DETAILED DESCRIPTION OF THE PREFERRED EMBODIMENTS

The present disclosure will be further described below with reference to the accompanying drawings and specific examples, such that those skilled in the art can better understand and implement the present disclosure. However, the examples given should not be construed as limiting the present disclosure.

Unless otherwise stated, the experimental methods used in the following examples are conventional methods. Unless otherwise stated, the materials, reagents, and the like used are commercially available.

The bottle chip used in the following examples is a recycled Nongfu Spring polyester bottle chip (Nongfu Spring drinking natural water).

The CAS numbers of the organic reagents used in the present disclosure are as follows:

TABLE 1

| Reagent | CAS |
|---|---|
| Polyethylene terephthalate | 25038-59-9 |
| Polypropylene terephthalate | 26590-75-0 |
| Polybutylene terephthalate | 26062-94-2 |
| Polyethylene 2,5- | 28728-19-0 |
| furandicarboxylate | |
| DMI | 80-73-9 |
| DABCO | 280-57-9 |
| Deuterated trichloromethane | 865-49-6 |
| Deuterated dichloromethane | 1665-00-5 |
| DBN | 3001-72-7 |
| TBD | 5807-14-7 |
| DBU | 6674-22-2 |
| TMG | 80-70-6 |
| MTBD | 84030-20-6 |
| 1,3-Bis(2,6- | 250285-32-6 |
| diisopropylphenyl) | |
| imidazoliumchloride | |
| NMI | 616-47-7 |
| DMAP | 1122-58-3 |
| 4-PPy | 2456-81-7 |
| BEMP | 98015-45-3 |
| DMPU | 7226-23-5 |
| NMP | 872-50-4 |
| n-Tetrabutyl titanate | 5593-70-4 |
| Dibutyltin oxide | 818-08-6 |
| Zinc acetate | 557-34-6 |

### Example 1

To a 250 mL three-necked flask were added 1.5 g of polyethylene terephthalate (PET), 120 mL of 1,3-dimethyl-2-imidazolinone (DMI), and 0.045 g of DABCO, and the resulting mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 62%.

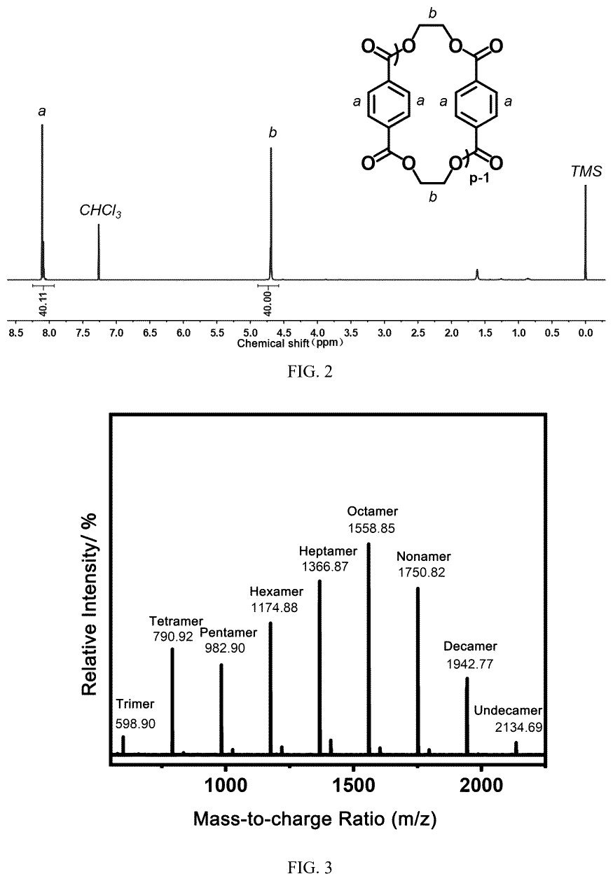

The chemical structure of the cyclic oligomeric polyethylene terephthalate was characterized by quantitative nuclear magnetic resonance hydrogen spectrum using deuterated trichloromethane as a solvent. The ¹H NMR spectra is shown in FIG. 2. The peak a was assigned to hydrogen on the benzene ring, and the peak b was assigned to the proton peak of methylene in the ethylene glycol chain segment. The test results demonstrate the successful synthesis of cyclic oligomeric polyethylene terephthalate.

The product was tested by matrix-assisted laser desorption ionization time of flight mass spectrometry (MALDI-TOF). The MALDI-TOF MS spectrum is shown in FIG. 3. The results show that molecular ion peaks appeared at 598.90 (m/z), 790.92 (m/z), 982.90 (m/z), 1174.88 (m/z), 1366.87 (m/z), 1558.85 (m/z), 1750.82 (m/z), 1942.77 (m/z), and 2134.69 (m/z), which corresponded to the sodium adduct ion peaks of the cyclic oligomeric polyethylene terephthalate trimer, tetramer, pentamer, hexamer, heptamer, octamer, nonamer, decamer and undecamer, respectively, and the measured mass-to-charge ratios of the peaks agreed with the theoretical values of 599.12 (m/z), 791.16 (m/z), 983.20 (m/z), 1175.24 (m/z), 1367.29 (m/z), 1559.33 (m/z), 1751.37 (m/z), 1943.41 (m/z), and 2135.45 (m/z). The test results demonstrate the successful synthesis of cyclic oligomeric polyethylene terephthalate, and the resulting product is a mixture of cyclic oligomeric polyethylene terephthalate with repeating units 3-11.

### Example 2

To a 250 mL three-necked flask were added 1.5 g of polyethylene terephthalate (PET), 120 mL of DMI, and 0.045 g of DBN, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 66%.

### Example 3

To a 250 mL three-necked flask were added 1.5 g of polyethylene terephthalate (PET), 120 mL of DMI, and 0.045 g of TBD, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 67%.

### Example 4

To a 250 mL three-necked flask were added 1.5 g of polyethylene terephthalate (PET), 120 mL of DMI, and 0.045 g of DBU, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 72%.

### Example 5

To a 250 mL three-necked flask were added 1.5 g of polyethylene terephthalate (PET), 120 mL of DMI, and 0.045 g of TMG, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 76%.

### Example 6

To a 250 mL three-necked flask were added 1.5 g of polyethylene terephthalate (PET), 120 mL of DMI, and 0.045 g of MTBD, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 77%.

### Example 7

To a 250 mL three-necked flask were added 1.5 g of polyethylene terephthalate (PET), 120 mL of DMI, and 0.045 g of 1,3-bis(2,6-diisopropylphenyl) imidazole chloride, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 77%.

### Example 8

To a 250 mL three-necked flask were added 1.5 g of polyethylene terephthalate (PET), 120 mL of DMI, and 0.045 g of NMI, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 80%.

### Example 9

To a 250 mL three-necked flask were added 1.5 g of polyethylene terephthalate (PET), 120 mL of DMI, and 0.045 g of DMAP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 83%.

### Example 10

To a 250 mL three-necked flask were added 1.5 g of polyethylene terephthalate (PET), 120 mL of DMI, and 0.045 g of 4-PPy, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 84%.

### Example 11

To a 250 mL three-necked flask were added 1.5 g of polyethylene terephthalate (PET), 120 mL of DMI, and 0.045 g of BEMP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 87%.

### Example 12

To a 250 mL three-necked flask were added 1.5 g of polyethylene terephthalate (PET), 120 mL of DMI, and 0.0075 g of DMAP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 58%.

### Example 13

To a 250 mL three-necked flask were added 1.5 g of polyethylene terephthalate (PET), 120 mL of DMI, and 0.060 g of DMAP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 85%.

### Example 14

To a 250 mL three-necked flask were added 1.5 g of polyethylene terephthalate (PET), 200 mL of DMI, and 0.045 g of DMAP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 87%.

### Example 15

To a 250 mL three-necked flask were added 1.5 g of polyethylene terephthalate (PET), 200 mL of DMI, and 0.045 g of BEMP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 91%.

### Example 16

To a 250 mL three-necked flask were added 1.5 g of polyethylene terephthalate (PET), 30 mL of DMI, and 0.045 g of DMAP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 49%.

### Example 17

To a 250 mL three-necked flask were added 1.5 g of polyethylene terephthalate (PET), 120 mL of DMI, and 0.045 g of DMAP, and the mixture was purged with nitrogen, heated, stirred for dissolving, and refluxed at 220° C. for 4 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 48%.

### Example 18

To a 250 mL three-necked flask were added 1.5 g of polyethylene terephthalate (PET), 120 mL of DMI, and 0.045 g of DMAP, and the mixture was purged with nitrogen, heated, stirred for dissolving, and refluxed at 220° C. for 16 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 80%.

### Example 19

To a 250 mL three-necked flask were added 1.5 g of polyethylene terephthalate (PET), 120 mL of DMI, and 0.045 g of DMAP, and the mixture was purged with nitrogen, heated, stirred for dissolving, and refluxed at 180° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 15%.

### Example 20

To a 250 mL three-necked flask were added 1.5 g of polyethylene terephthalate (PET), 120 mL of 1,3-dimethyl-3,4,5,6-tetrahydro-2-pyrimidone (DMPU), and 0.045 g of DMAP, and the mixture was purged with nitrogen, heated, stirred for dissolving, and refluxed at 240° C. for 4 h. After the reaction was completed, the mixture was distilled under reduced pressure (about 100 Pa, 100° C.) to remove the solvent, subjected to column chromatography to remove linear oligomers, settled in petroleum ether, and dried in vacuum to obtain cyclic oligomeric polyethylene terephthalate. The product was a white powder with a yield of 75%.

### Example 21

To a 250 mL three-necked flask were added 1.5 g of polyethylene terephthalate (PET), 120 mL of DMPU, and 0.045 g of DMAP, and the mixture was purged with nitrogen, heated, stirred for dissolving, and refluxed at 220° C. for 12 h. After the reaction was completed, the mixture was distilled under reduced pressure (about 100 Pa, 100° C.) to remove the solvent, subjected to column chromatography to remove linear oligomers, settled in petroleum ether, and dried in vacuum to obtain cyclic oligomeric polyethylene terephthalate. The product was a white powder with a yield of 75%.

### Example 22

To a 250 mL three-necked flask were added 1.5 g of polyethylene terephthalate (PET), 120 mL of N-methylpyrrolidone (NMP), and 0.045 g of DMAP, and the mixture was purged with nitrogen, heated, stirred for dissolving, and refluxed at 200° C. for 12 h. After the reaction was completed, the mixture was distilled under reduced pressure (about 100 Pa, 85° C.) to remove the solvent, subjected to column chromatography to remove linear oligomers, settled in petroleum ether, and dried in vacuum to obtain cyclic oligomeric polyethylene terephthalate. The product was a white powder with a yield of 47%.

### Example 23

To a 250 mL three-necked flask were added 1.5 g of polypropylene terephthalate (PTT), 120 mL of DMI, and 0.045 g of DMAP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polypropylene terephthalate. The product was a white powder with a yield of 72%.

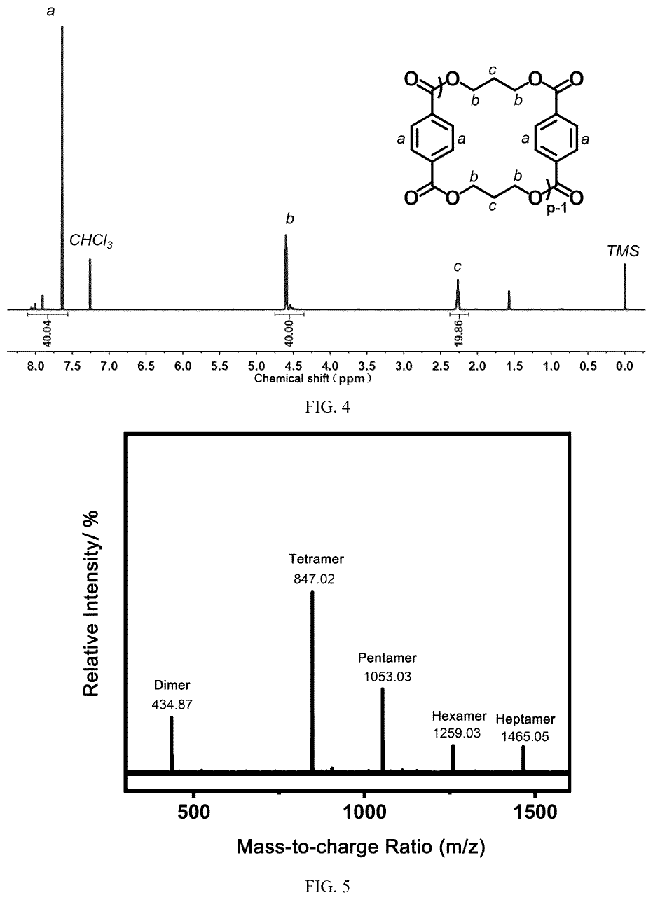

The chemical structure of the cyclic oligomeric polypropylene terephthalate was characterized by quantitative nuclear magnetic resonance hydrogen spectrum using deuterated trichloromethane as a solvent. The ¹H NMR spectra is shown in FIG. 4. The peak a was assigned to hydrogen on the benzene ring, the peak b was assigned to the proton peak of methylene of the propylene glycol chain segment near the ester bond, and the peak c was assigned to the proton peak of methylene in the middle of the propylene glycol chain segment. The test results demonstrate the successful synthesis of cyclic oligomeric polypropylene terephthalate.

The product was tested by matrix-assisted laser desorption ionization time of flight mass spectrometry (MALDI-TOF). The MALDI-TOF MS spectrum is shown in FIG. 5. The results show that molecular ion peaks appeared at 434.87 (m/z), 847.02 (m/z), 1053.03 (m/z), 1259.03 (m/z), and 1465.05 (m/z), which corresponded to the sodium adduction peaks of the cyclic oligomeric polypropylene terephthalate dimer, tetramer, pentamer, hexamer, and heptamer, respectively, and the measured mass-to-charge ratios of the peaks conformed with the theoretical values of 435.11 (m/z), 847.22 (m/z), 1053.28 (m/z), 1259.34 (m/z), and 1465.40 (m/z). The test results demonstrate the successful synthesis of cyclic oligomeric polypropylene terephthalate, the resulting product is a mixture of cyclic oligomeric polypropylene terephthalate with repeating units 2-7.

### Example 24

To a 250 mL three-necked flask were added 1.5 g of polybutylene terephthalate (PBT), 120 mL of DMI, and 0.045 g of DMAP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric butylene terephthalate. The product was a white powder with a yield of 61%.

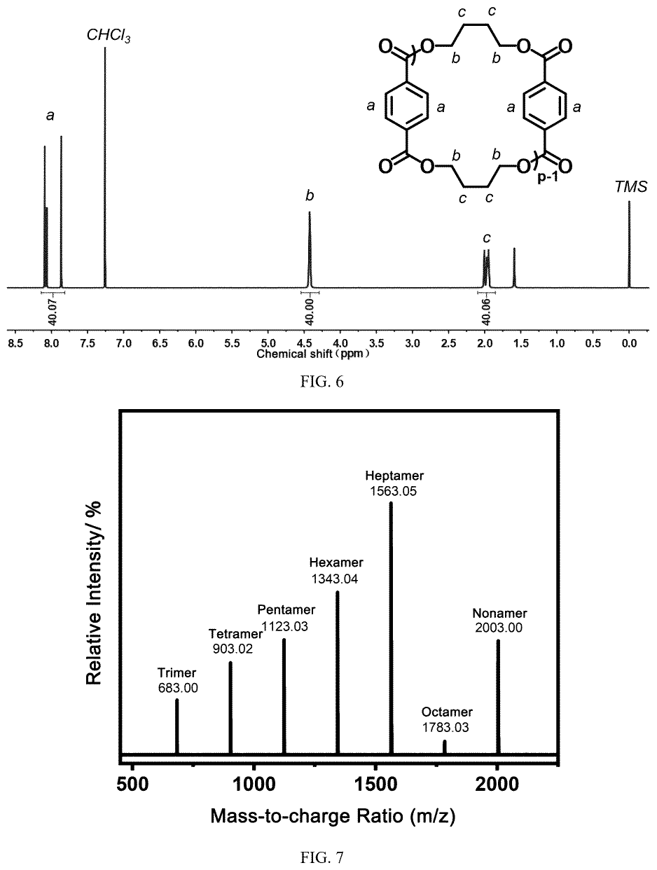

The chemical structure of the cyclic oligomeric butylene terephthalate was characterized by quantitative nuclear magnetic resonance hydrogen spectrum using deuterated trichloromethane as a solvent. The ¹H NMR spectra is shown in FIG. 6. The peak a was assigned to hydrogen on the benzene ring, the peak b was assigned to the proton peak of methylene of the butylene glycol chain segment near the ester bond, and the peak c was assigned to the proton peak of methylene in the middle of the butylene glycol chain segment. The test results demonstrate the successful synthesis of cyclic oligomeric butylene terephthalate.

The product was tested by matrix-assisted laser desorption ionization time of flight mass spectrometry (MALDI-TOF). The MALDI-TOF MS spectrum is shown in FIG. 7. The results show that molecular ion peaks appeared at 683.00 (m/z), 903.02 (m/z), 1123.03 (m/z), 1343.04 (m/z), 1563.05 (m/z), 1783.03 (m/z), and 2003.00 (m/z), which corresponded to the sodium adduction peaks of the cyclic oligomeric butylene terephthalate trimer, tetramer, pentamer, hexamer, heptamer, octamer and nonamer, respectively, and the measured mass-to-charge ratios of the peaks conformed with the theoretical values of 683.21 (m/z), 903.28 (m/z), 1123.36 (m/z), 1343.43 (m/z), 1563.50 (m/z), 1783.58 (m/z), and 2003.65 (m/z). The test results demonstrate the successful synthesis of cyclic oligomeric butylene terephthalate, the resulting product is a mixture of cyclic oligomeric butylene terephthalate with repeating units 3-9.

### Example 25

To a 250 mL three-necked flask were added 1.5 g of polyethylene furandicarboxylate (PEF), 120 mL of DMI, and 0.045 g of DMAP, and the mixture was purged with nitrogen, heated, stirred for dissolving, and refluxed at 200° C. for 10 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric ethylene furandicarboxylate. The product was a white powder with a yield of 67%.

The chemical structure of the cyclic oligomeric ethylene furandicarboxylate was characterized by quantitative nuclear magnetic resonance hydrogen spectrum using deuterated dichloromethane as a solvent. The ¹H NMR spectra is shown in FIG. 8. The peak a was assigned to hydrogen on the furan ring, and the peak b was assigned to the proton peak of methylene in the ethylene glycol chain segment. The test results demonstrate the successful synthesis of cyclic oligomeric ethylene furandicarboxylate.

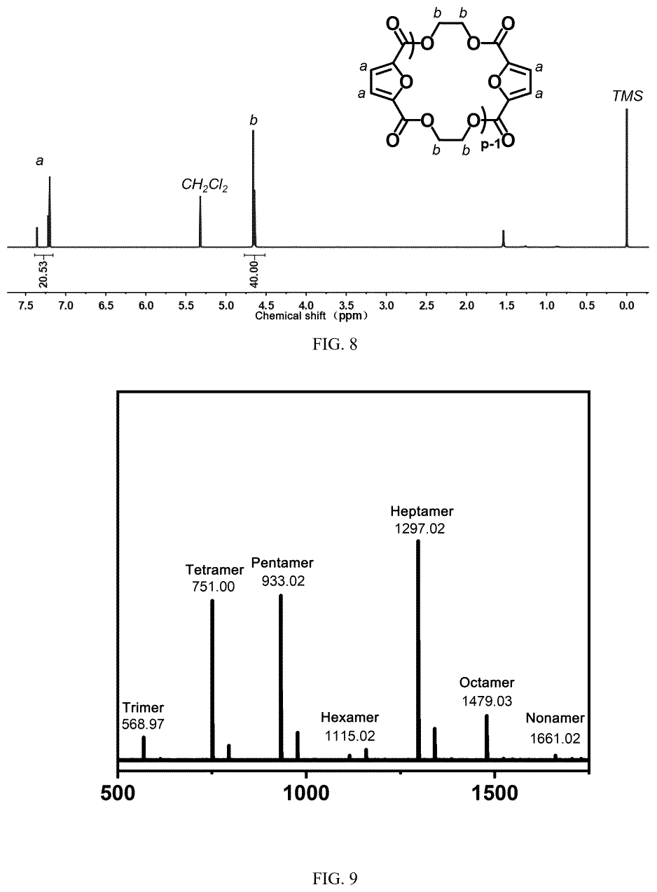

The product was tested by matrix-assisted laser desorption ionization time of flight mass spectrometry (MALDI-TOF). The MALDI-TOF MS spectrum is shown in FIG. 9. The results show that molecular ion peaks appeared at 568.97 (m/z), 751.00 (m/z), 933.02 (m/z), 1115.02 (m/z), 1297.02 (m/z), 1479.03 (m/z), and 1661.02 (m/z), which corresponded to the sodium adduction peaks of the cyclic oligomeric ethylene furandicarboxylate trimer, tetramer, pentamer, hexamer, heptamer, octamer and nonamer, respectively, and the measured mass-to-charge ratios of the peaks conformed with the theoretical values of 569.06 (m/z), 751.08 (m/z), 933.10 (m/z), 1115.12 (m/z), 1297.14 (m/z), 1479.16 (m/z), and 1661.18 (m/z). The test results demonstrate the successful synthesis of cyclic oligomeric ethylene furandicarboxylate, the resulting product is a mixture of cyclic oligomeric ethylene furandicarboxylate with repeating units 3-9.

### Example 26

To a 250 mL three-necked flask were added 1.5 g of recycled Nongfu Spring PET bottle chips, 120 mL of DMI, and 0.045 g of DMAP, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 82%.

### Example 27

To a 100 mL three-necked flask were added 6 g of cyclic oligomeric polyethylene terephthalate (synthesized according to the method described in Example 26), which was purged with nitrogen, heated, and stirred to melt, and 3 mg of catalyst Sb₂O₃ was added. The mixture was reacted at 310° C. for 1.5 h to give a PET polyester. The intrinsic viscosity was measured by an Ubbelohde viscometer using a mixed solvent of phenol and tetrachloroethane (in a mass ratio of 6:4) at 25° C. and was found to be 0.60 dL/g, indicating that the cyclic oligoesters obtained by depolymerization can be used as monomers for repolymerization to obtain polyesters, realizing the closed-loop chemical recycling of polyesters.

### Comparative Example 1

To a 250 mL three-necked flask were added 1.5 g of polyethylene terephthalate (PET), 120 mL of DMI, and 0.045 g of n-tetrabutyl titanate, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 42%.

### Comparative Example 2

To a 250 mL three-necked flask were added 1.5 g of polyethylene terephthalate (PET), 120 mL of DMI, and 0.045 g of dibutyltin oxide, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 41%.

### Comparative Example 3

To a 250 mL three-necked flask were added 1.5 g of polyethylene terephthalate (PET), 120 mL of DMI, and 0.045 g of zinc acetate, and the mixture was purged with nitrogen, heated, stirred for dissolving, and refluxed at 220° C. for 12 h. After the reaction was completed, the mixture was distilled under reduced pressure (about 100 Pa, 90° C.) to remove the solvent, subjected to column chromatography to remove linear oligomers, settled in petroleum ether, and dried in vacuum to obtain cyclic oligomeric polyethylene terephthalate. The product was a white powder with a yield of 59%.

It is obvious that the above embodiments are merely examples for clear illustration and are not intended to limit implementations. Various changes and modifications can be made by those of ordinary skill in the art on the basis of the above description. It is unnecessary and impossible to exhaust all the embodiments herein. Obvious changes or modifications derived therefrom still fall within the scope of protection of the present disclosure.

What is claimed is:

1. A cyclodepolymerization method for an aromatic polyester, comprising steps of:
mixing an aromatic polyester with a high-boiling-point solvent and a catalyst to obtain a mixture, and heating and dissolving the mixture to subject the aromatic polyester to a cyclodepolymerization reaction to obtain aromatic cyclic oligoesters,
wherein the high-boiling-point solvent is selected from the group consisting of N-methylpyrrolidone, 1,3-dimethyl-2-imidazolinone, and 1,3-dimethyl-3,4,5,6-tetrahydro-2-pyrimidone and any combination thereof.

2. The cyclodepolymerization method according to claim 1, wherein the aromatic polyester is a recycled or synthetic aromatic polyester; the recycled aromatic polyester comprises a recycled bottle chip and/or a recycled fiber.

3. The cyclodepolymerization method according to claim 2, wherein the aromatic polyester is formed by polymerization of an aromatic diacid and an alkylene glycol, and has a structural formula of: 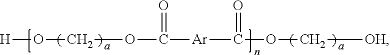
wherein Ar is an aromatic ring; a=2-4, and a is an integer; n≥4, and n is an integer.

4. The cyclodepolymerization method according to claim 3, wherein the aromatic ring is a benzene ring and/or a furan ring; the alkylene glycol is selected from the group consisting of ethylene glycol, 1,3-propanediol, 1,4-butanediol and any combination thereof.

5. The cyclodepolymerization method according to claim 1, wherein the aromatic cyclic oligoesters have a structural formula of: 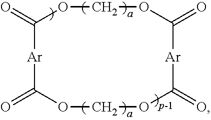
wherein Ar is an aromatic ring, a=2-4, p=2-13, and an integer is for both a and p.

6. The cyclodepolymerization method according to claim 1, wherein the catalyst is an organic base catalyst; the organic base catalyst is selected from the group consisting of an alkylamine catalyst, a pyridine derivative catalyst, an amidine catalyst, a guanidine catalyst, an azacyclic carbene catalyst, a phosphonitrile catalyst and any combination thereof.

7. The cyclodepolymerization method according to claim 1, wherein the catalyst is added in an amount of 0.1%-10% by mass of the aromatic polyester.

8. The cyclodepolymerization method according to claim 1, wherein the aromatic polyester has a concentration of 1 g/L to 100 g/L in the mixture.

9. The cyclodepolymerization method according to claim 1, wherein the reaction time is 3 h-24 h; the heating temperature is 180° C.-250° C.

10. The cyclodepolymerization method according to claim 1, wherein the aromatic cyclic oligoesters obtained by cyclodepolymerization are further polymerized as monomers to produce an aromatic polyester.
